(19) United States
(12) Patent Application Publication
(10) Pub. No.: US 2012/0172737 A1
(43) Pub. Date: Jul. 5, 2012

(54) Measurement Device, Measurement System and Data Processing Method for Physiological signal

(76) Inventors: Pai-Yang Lin, New Taipei City (TW); Yao-Tsung Chang, New Taipei City (TW); Chia-Hsien Li, New Taipei City (TW); Shun-Chi Chung, New Taipei City (TW); Shu-Hung Lin, New Taipei City (TW)

(21) Appl. No.: 13/291,066

(22) Filed: Nov. 7, 2011

(30) Foreign Application Priority Data
Dec. 31, 2010 (TW) 099147199

(51) Int. Cl. A61B 5/0402; A61B 5/00

(52) U.S. Cl. 600/509; 600/300

(57) ABSTRACT

A measurement device for measuring a physiological signal of a testee includes a sensing unit for sensing physiological activities of the testee to generate the physiological signal, a data transmission unit for transmitting data to a host, a storage unit for storing data, and a control unit for deciding to use the data transmission unit to transmit the physiological signal to the host or to use the storage unit to store the physiological signal according to a connection status between the data transmission unit and the host.

### BACKGROUND OF THE INVENTION

1. Field of the Invention

The present invention relates to a measurement device for a physiological signal, measurement system and data processing method, and more particularly, to a measurement device, measurement system and data processing method capable of enhancing facility and flexibility of measuring a physiological signal.

2. Description of the Prior Art

Advances in electrical and electronic technologies not only boost work efficiency, but also help maintain health and wellbeing. Electrocardiography is one such example, which records physiological activities of a heart over time, enabling doctors to determine cardiovascular conditions of a testee, and allowing early diagnosis and treatment for related disorders.

Traditionally, electrocardiograph monitoring devices can be divided into two major types. Firstly, a clinical type is a large medical apparatus found in health institutions, which obtains physiological signals through disposable skin electrodes. Due to the bulk and operation complexity of such devices, professional medical personnel and a longer preparation time are required for making measurements, precluding domestic use by average users. The second is a portable handheld type, which makes measurements through a user contacting sensing elements on the measurement device with both hands, to obtain heart rate and electrocardiograph curves. However, other than its facility of measurements, the portable handheld type electrocardiograph measurement device has limited functionalities and built-in memory, thus large amounts of data cannot be stored locally but instead need to be transferred to a host (e.g. a computer) through USB or other interfaces for storage. Moreover, portability requirements limit the portable handheld type electrocardiograph measurement device to having only 2 to 4 inch displays, often causing font sizes and graphics to be too small for easy data reading. If data results are to be read via the host, the data must be transferred from the portable handheld type electrocardiograph measurement device to the host, and then displayed by the latter; in other words, the conventional portable handheld type electrocardiograph measurement device is incapable of making measurements and concurrently showing the results through the host, thus lacking convenience and flexibility.

As can be seen from the above, the conventional portable handheld type electrocardiograph measurement device, despite its portability, has only a fixed mode of operation, i.e. first making measurements, and then transferring data to the host, thus it is incapable of adaptively changing the operation mode to suit different situations, which is both inconvenient and inflexible.

### SUMMARY OF THE INVENTION

Therefore, the present invention mainly provides a measurement device for a physiological signal, measurement system and data processing method.

The present invention discloses a measurement device, for measuring a physiological signal of a testee, which comprises a sensing unit, for sensing physiological activities of the testee, to generate the physiological signal, a data transmission unit, for transmitting data to a host, a storage unit, for storing data, and a control unit, for deciding to use the data transmission unit to transmit the physiological signal to the host or use the storage unit to store the physiological signal according to a connection status between the data transmission unit and the host.

The present invention further discloses a measurement system, for measuring a physiological signal of a testee, which comprises a host and a measurement device. The host comprises a data receiving unit, for receiving the physiological signal, and an analyzing unit, for analyzing the physiological signal received by the data receiving unit. The measurement device comprises a sensing unit, for sensing physiological activities of the testee, to generate the physiological signal, a data transmission unit, for transmitting data to the data receiving unit, a storage unit, for storing data, and a control unit, for deciding to use the data transmission unit to transmit the physiological signal to the host or use the storage unit to store the physiological signal according to a connection status between the data transmission unit and the host.

The present invention further discloses a data processing method for a measurement system. The measurement system comprises a measurement device and a host, for measuring a physiological signal of a testee. The data processing method comprises the measurement device sensing physiological activities of the testee, to generate the physiological signal, and the measurement device deciding to transmit the physiological signal sensed by the measurement device to the host, or to store the physiological signal in the measurement device according to a connection status between the measurement device and the host.

These and other objectives of the present invention will no doubt become obvious to those of ordinary skill in the art after reading the following detailed description of the preferred embodiment that is illustrated in the various figures and drawings.

### BRIEF DESCRIPTION OF THE DRAWINGS

FIG. 1 is a schematic diagram for a measurement system according to an embodiment of the present invention.

FIG. 2 is a schematic diagram for a measurement system according to another embodiment of the present invention.

FIG. 3 is a schematic diagram for a data processing process according to an embodiment of the present invention.

### DETAILED DESCRIPTION

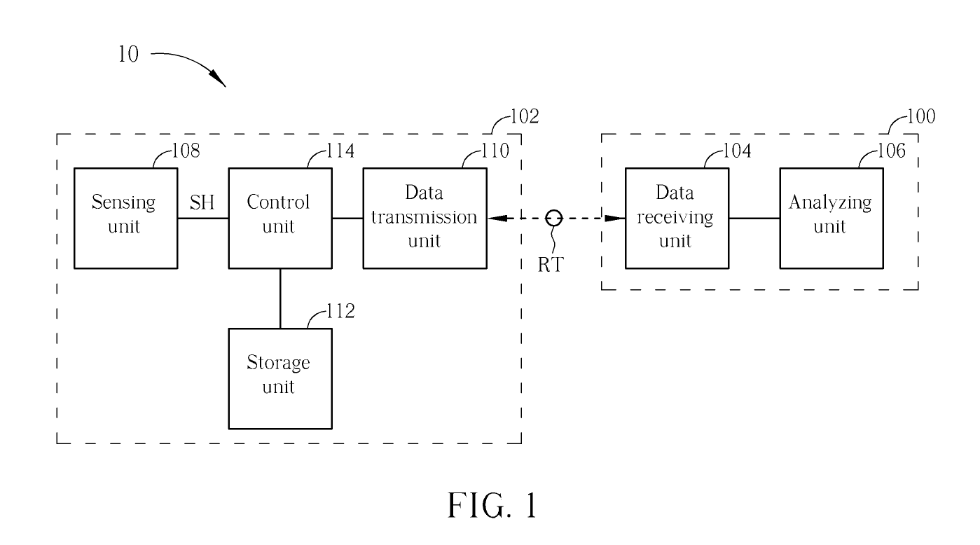

Please refer to FIG. 1, which is a schematic diagram of a measurement system 10 according to an embodiment of the present invention. The measurement system 10 comprises a host 100 and a measurement device 102, for sensing physiological activities of a testee, to generate a corresponding physiological signal SH. The measurement device 102 can adaptively change an operation mode according to a connection status between the measurement device 102 and the host 100, to enhance facility and flexibility. Note that, the measurement system 10 in FIG. 1 merely shows components related to the concept of the present invention, and other modifications may be made according to different requirements.

In the measurement system 10, the host 100 comprises a data receiving unit 104 and an analyzing unit 106, while the measurement device 102 comprises a sensing unit 108, a data transmission unit 110, a storage unit 112 and a control unit 114. The sensing unit 108 senses physiological activities of the testee, to generate the physiological signal SH. The control unit 114 decides to use the data transmission unit 110 to transmit the physiological signal sensed by the sensing unit 108 to the receiving unit 104, or use the storage unit 112 to store the physiological signal SH according to the connection status between the data transmission unit 110 and the receiving unit 104. Specifically, in FIG. 1, a dotted line RT is drawn between the data transmission unit 110 and the data receiving unit 104 to represent the connection status between the data transmission unit 110 and the data receiving unit 104 is not a fixed connection, and that signal connection between the data transmission unit 110 and the data receiving unit 104 may either be established or not established. If signal connection is established between the data transmission unit 110 and the data receiving unit 104, the control unit 114 transmits the physiological signal SH sensed by the sensing unit 108 through the data transmission unit 110 to the data receiving unit 104, such that the analyzing unit 106 can instantly analyze the physiological signal SH received by the data receiving unit 104, and in turn display corresponding indication signals or graphs. Conversely, if signal connection is not established between the data transmission unit 110 and the data receiving unit 104, the control unit 114 stores the physiological signal SH sensed by the sensing unit 108 in the storage unit 112, and later transmit the physiological signal SH to the data receiving unit 104 once signal connection is established between the data transmission unit 110 and the data receiving unit 104. As can be seen, the measurement device 102 can implement different modes of operation (e.g. traditional clinical type or portable handheld type measurement device) according to the connection status between the measurement device 102 and the host 100.

Simply put, the essential concept of the present invention is adaptively adjusting modes of operation of the measurement device 102 according to different connection statuses between the host 100 and the measurement device 102, to enhance convenience and flexibility of operation. In other words, if an operator of the measurement system 10 intends to instantly read the physiological signal SH of the testee, the operator only needs to use the data transmission unit 110 to establish the signal connection to the data receiving unit 104, and use the sensing unit 108 of the measurement device 102 to measure the physiological signal SH of the testee. As a result, the measurement device 102 automatically transmits the measured physiological signal SH to the data receiving unit 104 instantly, and the analyzing unit 106 analyzes and outputs the corresponding indication signals or graphs. In this way, the operator is able to access the physiological readings of the testee directly via the host 100. Alternatively, when signal connection is not established between the data transmission unit 110 and the data receiving unit 104, as is the case if the testee wishes to independently measure and record the physiological signal SH at home or outdoors, the measurement device 102 stores the physiological signal SH measured by the sensing unit 108 in the storage unit 112, to await the establishment of the signal connection between the data transmission unit 110 and the data receiving unit 104, before transmitting the data in the storage unit 112 to the host 100.

Therefore, the measurement system 10 is capable of adaptively adjusting the operation mode of the measurement device 102 according to different connection statuses. It should be noted that the measurement system 10 in FIG. 1 merely illustrates a concept of the present invention, and that any modifications made based on this concept are within the scope of the present invention. For instance, the physiological signal SH may be an electrocardiograph waveform signal, numerical data, etc. and is not limited thereto. Or, the physiological signal SH may also be any other kind of quantitatively measurable physiological data, e.g. heart rate, blood pressure, blood sugar level, body temperature, etc. The measurement device 102 may include a display unit, for displaying the physiological signal SH measured by the sensing unit 108, or corresponding graph forms, numerical values, etc of the physiological signal SH. Additionally, the measurement device 102 may also include an indication unit, for indicating the storage status of the storage unit 112, e.g. to notify the operator of insufficient storage space in the storage unit 112 with an indication light or sound alert. Similarly, the host 100 may also include a display unit, to display analysis results from the analyzing unit 106, or a storage unit to store analysis results of the analyzing unit 106. Furthermore, different technologies may be employed to establish the signal connection between the data transmission unit 110 and the data receiving unit 104, e.g. wireless (Bluetooth, infrared, RF, etc.) or wired (USB, IEEE 1394, etc.), so long as both sides employ the same communication technology and the connection can be successfully established.

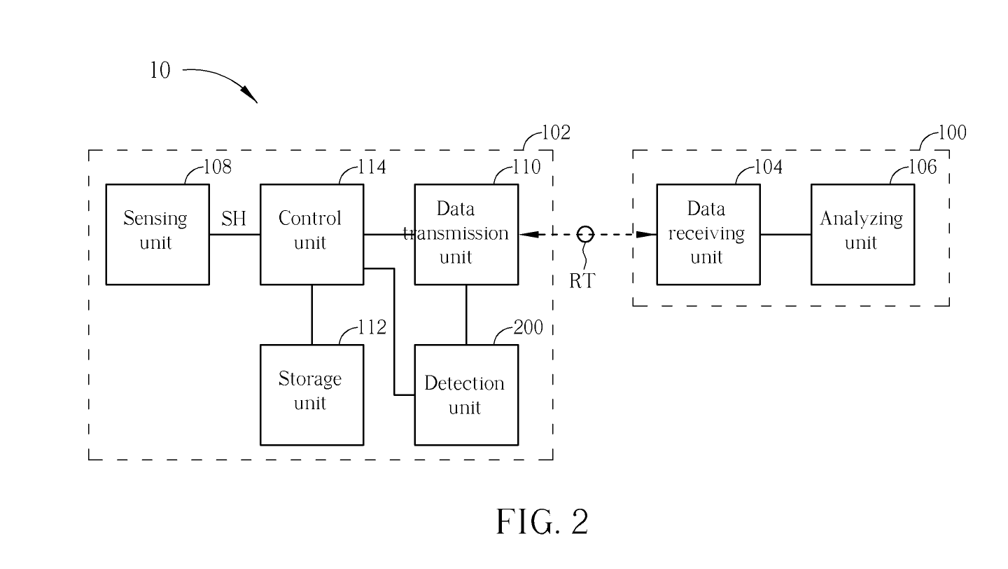

Apart from hardware modifications, operation of the measurement system 10 is also open to variations, e.g. the measurement device 102 may operate in a hibernation mode when not in the process of measuring or after no measurements have taken place for a predefined continuous time duration, when the testee touches the sensing unit 108, the sensing unit 108 is activated and starts sensing physiological activities of the testee. Also, detection of the connection status between the data transmission unit 110 and the data receiving unit 104 does not necessarily need to be executed by the control unit 114. Alternatively, as shown in FIG. 2, an additional detection unit 200 detects the connection status between the data transmission unit 110 and the data receiving unit 104, and then returns the detected results back to the control unit 114, to determine whether to transmit the physiological signal SH to the host 100 or to store it to storage unit 112.

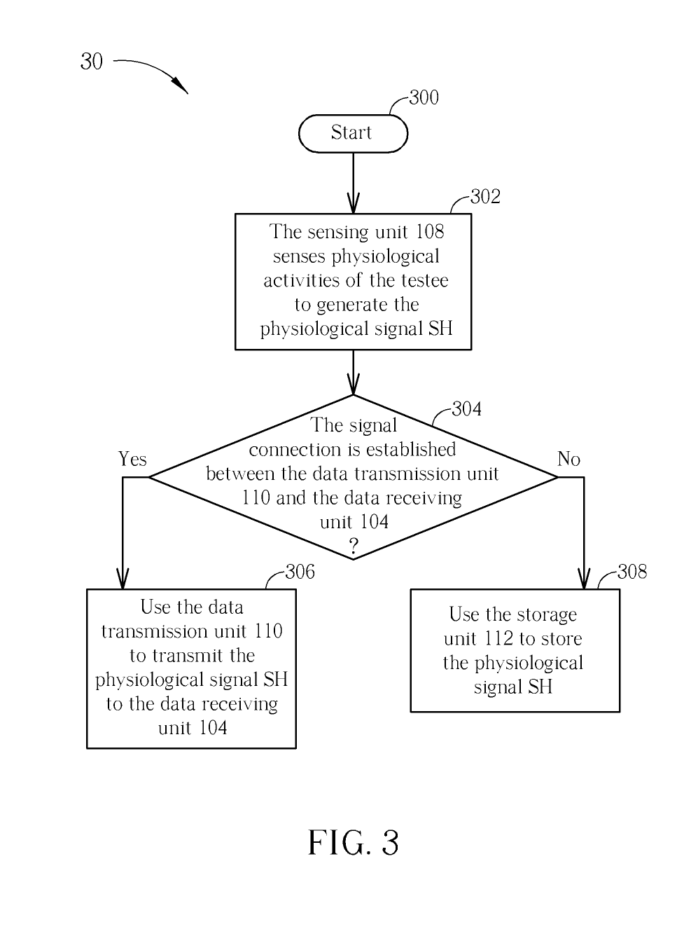

Aforementioned modifications are to emphasize the essential concept of the present invention that the measurement system 10 may adjust its operation mode according to different connection statuses between the host 100 and the measurement device 102, to adaptively implement clinical or portable handheld type measurement devices. Operations of the measurement device 102 on the physiological signal SH according to the connection status between the host 100 and the measurement device 102 can be summarized into a data processing process 30, as shown in FIG. 3. The data processing process 30 comprises the following steps:
step 300: Start.
step 302: The sensing unit 108 senses physiological activities of the testee to generate the physiological signal SH.
step 304: The control unit 114 determines a signal connection status between the data transmission unit 110 and the data receiving unit 104. If the signal connection is established between the data transmission unit 110 and the data receiving unit 104, go to step 306; else if the signal connection is not established between the data transmission unit 110 and the data receiving unit 104, go to step 308.
step 306: Use the data transmission unit 110 to transmit the physiological signal SH to the data receiving unit 104.
step 308: Use the storage unit 112 to store the physiological signal SH.

Details and modifications of the data processing process 30 can be found in aforementioned descriptions, and are not reiterated here.

In the prior art, a portable handheld type measurement device facilitates portability, but is limited to a fixed operation mode, i.e. first measuring, then transmitting the measured data to the host, thus is incapable of adaptively adjusting operation modes for different situations and lacks convenience and flexibility. Comparatively, the measurement system 10 in the present invention adjusts operation modes of the measurement device 102 according to the connection status between the host 100 and the measurement device 102, to adaptively implement clinical or portable handheld type measurement devices, thus enhancing facility and flexibility.

In summary, the present invention adjusts operation modes of the measurement device according to the connection status between the measurement device and the host, to enhance convenience and flexibility.

Those skilled in the art will readily observe that numerous modifications and alterations of the device and method may be made while retaining the teachings of the invention.

What is claimed is:

1. A measurement device, for measuring a physiological signal of a testee, comprising:
a sensing unit, for sensing physiological activities of the testee, to generate the physiological signal;
a data transmission unit, for transmitting data to a host;
a storage unit, for storing data; and
a control unit, for deciding to use the data transmission unit to transmit the physiological signal to the host or to use the storage unit to store the physiological signal according to a connection status between the data transmission unit and the host.

2. The measurement device of claim 1, wherein the physiological signal corresponds to an electrocardiograph.

3. The measurement device of claim 1, wherein the control unit uses the data transmission unit to transmit the physiological signal to the host when signal connection is established between the data transmission unit and the host.

4. The measurement device of claim 3, wherein the control unit further uses the data transmission unit to transmit data stored in the storage unit to the host when the signal connection is established between the data transmission unit and the host.

5. The measurement device of claim 1, wherein the control unit uses the storage unit to store the physiological signal when signal connection is not established between the data transmission unit and the host.

6. A measurement system for measuring a physiological signal of a testee, comprising:
a host, comprising:
a data receiving unit, for receiving the physiological signal; and
an analyzing unit, for analyzing the physiological signal received by the data receiving unit; and
a measurement device, comprising:
a sensing unit, for sensing physiological activities of the testee to generate the physiological signal;
a data transmission unit, for transmitting data to the data receiving unit;
a storage unit, for storing data; and
a control unit, for deciding to use the data transmission unit to transmit the physiological signal to the data receiving unit or to use the storage unit to store the physiological signal according to a connection status between the data transmission unit and the data receiving unit.

7. The measurement system of claim 6, wherein the physiological signal corresponds to an electrocardiograph.

8. The measurement system of claim 6, wherein the control unit uses the data transmission unit to transmit the physiological signal to the data receiving unit when signal connection is established between the data transmission unit and the data receiving unit.

9. The measurement system of claim 8, wherein the control unit further uses the data transmission unit to transmit the data stored in the storage unit to the data receiving unit when the signal connection is established between the data transmission unit and the data receiving unit.

10. The measurement system of claim 6, wherein the control unit uses the storage unit to store the physiological signal when signal connection is not established between the data transmission unit and the data receiving unit.

11. A data processing method for a measurement system, the measurement system comprising a measurement device and a host, for measuring a physiological signal of a testee, the data processing method comprising:
the measurement device sensing physiological activities of the testee to generate the physiological signal; and
the measurement device deciding to transmit the physiological signal sensed by the measurement device to the host or to store the physiological signal into the measurement device according to a connection status between the measurement device and the host.

12. The data processing method of claim 11, wherein the physiological signal corresponds to an electrocardiograph.

13. The data processing method of claim 11, wherein the step of the measurement device deciding to transmit the physiological signal sensed by the measurement device to the host or to store the physiological signal into the measurement device according to the connection status between the measurement device and the host comprises transmitting the physiological signal sensed by the measurement device to the host when signal connection is established between the measurement device and the host.

14. The data processing method of claim 13, further comprising the measurement device transmitting the data stored in the storage unit to the host when the signal connection is established between the measurement device and the host.

15. The data processing method of claim 11, wherein the step of the measurement device deciding to transmit the physiological signal sensed by the measurement device to the host or to store the physiological signal into the measurement device according to the connection status between the measurement device and the host comprises the measurement device storing the physiological signal into the measurement device when signal connection is not established between the measurement device and the host.
